(19) United States
(12) Patent Application Publication
(10) Pub. No.: US 2024/0050588 A1
(43) Pub. Date: Feb. 15, 2024

(54) PHARMACEUTICAL COMPOSITION CONTAINING PROPOFOL, A CYCLODEXTRIN OR A CYCLODEXTRIN DERIVATIVE AND A PHARMACEUTICALLY ACCEPTABLE SALT

(71) Applicant: CENTRE HOSPITALIER UNIVERSITAIRE DE LILLE, Lille (FR)

(72) Inventors: Myriam BOUCHFAA, Beaucamps Ligny (FR); Damien ROUSSELEAU, Lille (FR); Damien LANNOY, Wattignies (FR); Pascal ODOU, Godewaersvelde (FR)

(21) Appl. No.: 18/256,719

(22) PCT Filed: Dec. 10, 2021
(86) PCT No.: PCT/EP2021/085133

§ 371 Date: Jun. 9, 2023

(30) Foreign Application Priority Data
Dec. 10, 2020 (FR) 2012996

(51) Int. Cl. A61K 47/69; A61K 31/05; A61K 47/02

(52) U.S. Cl. CPC A61K 47/6951; A61K 31/05; A61K 47/02

(57) ABSTRACT

The present invention relates to a pharmaceutical composition having propofol and at least one cyclodextrin and/or a cyclodextrin derivative. Characteristically, according to the invention, it further includes at least one pharmaceutically acceptable salt, with the exception of basic and/or acidic pharmaceutically acceptable salts.

### BACKGROUND

Propofol is a highly liposoluble compound. Hence, it is nowadays essentially administered in the form of a lipophilic-in-hydrophilic (oil-in-water) emulsion. The emulsions available on the market are always formed using soybean oil, phospholipids extracted from eggs and glycerol. This emulsion is sterilised by terminal sterilisation, and not by terminal filtration, the globules having an unsuitable average size to be able to pass through the filter. Since sterilisation has to be carried out in a rotary mode, the preparation of the emulsion can therefore be carried out only industrially.

The Injection of propofol remains painful because of the free propofol within the emulsion.

### DISCUSSION OF PRIOR ART

The document CA 2 474 710 A relates to a pharmaceutical composition containing a complex of propofol and a water-soluble cyclodextrin, 2-hydroxy-propyl-beta-cyclodextrin. This composition is in a freeze-dried form. The propofol:cyclodextrin molar ratio is 1:>1 in particular 1:1.5 to 1:2. Preferably, the degree of substitution of 2-hydroxypropyl-beta-cyclodextrin is between 2.5 and 9.0 and more preferably between 4.6 and 5.1 of 2-hydroxypropyl groups per beta-cyclodextrin molecule. If this value is related to the number of glucopyranose units in the beta-cyclodextrin, a degree of substitution of 0.35 to 1.28, preferably 0.66 to 0.73, is obtained (the beta-cyclodextrin contains 7 glucopyranose units). In the experimental examples of the aforementioned document, the used average degree of substitution is equal to 4.6, namely 0.657 when related to the number of glucopyranose units.

The document U.S. Pat. No. 7,034,013 B2 relates to a liquid pharmaceutical composition which comprises propofol, a sulpho alkyl ether of cyclodextrin and a liquid excipient. Cyclodextrin ether has less haemolytic power than hydroxypropyl-β-cyclodextrins. More generally, it describes liquid and transparent compositions of propofol and a cyclodextrin alkyl ether named Captisol®. The molar ratio of sulpho ethyl ether: propofol ranges from 1:1 to 5:1 which indicates that a propofol molecule is complexed with one or more molecule(s) of the sulphurised alkyl ether of cyclodextrin.

The document U.S. Pat. No. 7,138,387 B2 describes a pharmaceutical composition of propofol and 2-hydroxypropyl-beta-cyclodextrin whose mass ratio propofol:HPBCD (hydropropyl-β-cylodextrin) is from 1:30 to 1:60 which corresponds to a molar ratio of 1:3 to 1:7. This composition may be sterilised by autoclaving. The degree of substitution of the HPBCD is not indicated. This composition contains a high dose of 2-hydroxypropyl-beta-cyclodextrin, which is nephrotoxic like the sodium salt of sulphobutyl-ether of beta-cyclodextrin in case of prolonged administration.

The publication entitled “Evaluation of new propofol aqueous solutions for intravenous anesthesia”, by Trapani, A. et al. and published in Int. J.Pharm. 278, 91-98 in 2004 describes compositions containing 2-hydropropyl-β-cyclodextrin whose degree of substitution is equal to 5.88 (which corresponds to an average substitution rate per glucopyranose unit equal to 0.84), water and propofol.

The publication by Wallentine, C. B., Shimode, N., Egan, T. D. & Pace, N. L. entitled “Propofol in a modified cyclodextrin formulation: First human study of dose-response with emphasis on injection pain” and published in the journal Anesth. Analg. 113, 738-741 in 2011 describes a composition of propofol and sulpho-butyl ether-beta-cyclodextrin (Captisol®) in water, without information on the formulation, the manufacturing process, the amount of cyclodextrin derivative, the pH or the osmolality. This publication concludes that the injection of this solution is no less painful than that of propofol in emulsion. These results are based on a small-sized sample of patients.

The publication by Trapani, G. et al. entitled “Inclusion complexation of propofol with 2-hydroxypropyl-β-cyclodextrin Physicochemical, nuclear magnetic resonance spectroscopic studies, and anesthetic properties in rat” and published in the journal J. Pharm. Sci. 87, 514-518 in 1998 describes a solution of propofol complexed with 2-hydroxypropyl-beta-cyclodextrin, the average degree of substitution of which is 1.0, which corresponds to an average degree or rate of substitution per unit of glucopyranose in the hydroxypropyl-beta-cyclodextrin molecule equal to 0.14. This publication indicates that the propofol molecule is accommodates in the cavity formed by 2-hydroxypropyl-beta-cyclodextrin, the OH group of propofol projecting from this cavity.

The publication by Bielen, S. J., “ysko’ G. S. & Gough, W. B. entitled “The effect of a cyclodextrin vehicle on the cardiovascular profile of propofol in rats” and published in the journal Anesth. Analg. 82, 920-924 in 1996 describes compositions containing propofol and 2-hydroxypropyl-beta-cyclodextrin and probably water. The degree of substitution of 2-hydroxypropyl-beta-cyclodextrin is not indicated.

The document U.S. Pat. No. 9,006,216 B2 indicates that it is possible to solubilise the conjugate base of propofol, i.e. the ionic form of propofol which appears when the hydroxyl radical loses a proton. To obtain this base, the propofol has to be at a basic pH, in particular higher than 8 and preferably in the range of 8 to 11. We are then in the presence of two chemical balances: the acid-base balance of propofol and the balance of the complexation reaction of the base of propofol. Consequently, according to this document, the more basic the solution, the higher the concentration of the conjugate base of propofol and the more it is possible to solubilise the latter with 2-hydroxypropyl-beta-cyclodextrin. According to this document, the complexation (solubilisation) of propofol requires from 2 to 10 hours. The 2-hydroxypropyl-beta-cyclodextrin is first dissolved in a sodium hydroxide solution and then the propofol is added. The final composition has a pH of 9-10 and is not transparent since it has to be filtered by means of a filter with a pore size of 0.45 μm. 1 g of propofol has been mixed with 14.68 g of 2-hydroxypropyl-beta-cyclodextrin but because of the presence of insoluble matter removed by filtration, it is difficult to determine which mass of propofol has actually been complexed and therefore solubilised. Moreover, this solution cannot be sterilised by filtration because the pores of the sterilising filter are rapidly clogged with suspended matter. Moreover, the problem of the inaccuracy of the solubilised amount of propofol and of the basic pH makes the composition unusable in medicine and in particular for administration by the parenteral route.

### SUMMARY

An object of the present invention is to provide a new composition containing propofol and a cyclodextrin and/or a cyclodextrin derivative.

Another object of the present invention is to provide a composition which allows complexing a given amount of propofol with an optimised amount of cyclodextrin(s) and/or cyclodextrin derivative (s).

Another object of the present invention is to provide a propofol composition which can be administered by the parenteral route and more particularly by the intravascular route, i.e. injected into the blood system of a patient.

Another object of the present invention is to provide a method for manufacturing an injectable propofol composition which is simple and quick to implement.

Another object of the present invention is to provide an injectable composition of propofol which can be sterilised by filtration on a suitable membrane.

Another object of the present invention is to provide an injectable composition whose administration by the parenteral route is potentially less painful.

Another object of the present invention is to provide an injectable composition which is iso-osomolar and/or has a pH suitable for use by the intravascular route.

Another object of the present invention is to provide a composition which is stable over time.

The present invention relates to a pharmaceutical composition including propofol and at least one cyclodextrin and/or a cyclodextrin derivative according to claim 1 of the present application. Typically, according to the invention, it further comprises at least one pharmaceutically-acceptable salt, with the exception of basic and/or acidic pharmaceutically-acceptable salts.

### BRIEF DESCRIPTION OF THE FIGURES

FIG. 1 represents the 95% accuracy profile of the propofol assay method, i.e. it represents the error percentage of the method as a function of the amount of propofol measured in mL.

FIG. 2 represents the evolution of the concentration of dissolved propofol, calculated according to the method of Higuchi and Connors in mol of liquid composition as a function of the concentration (in mol) of each of the three 2-hydroxypropyl-β-cyclodextrins, which differ by the degree of substitution of the glucopyranose units;

FIG. 3 represents the solubilisation test of the cyclodextrin HPB-LB in the presence of different excipients according to the method of Higuchi and Connors; in the presence of different excipients, sodium chloride, magnesium chloride and glycerol, respectively.

### DETAILED DESCRIPTION

Indeed, it is the merit of the Inventors to have noticed that it was possible to optimise the amount of cyclodextrin/cyclodextrin derivative to solubilise the same given amount of propofol by adding a salt, in particular, a pharmaceutically-acceptable salt while maintaining a pH and an osmolality enabling the intravascular administration of the composition of the invention, when the latter is in the form of a solution. Without the Inventors being bound by the following explanation, it seems that the presence of ions in the solution modifies the dielectric constant of the solution; the modification of this dielectric constant modifies the balance between the amount of complexed propofol, the amount of solvated cyclodextrin or cyclodextrin derivative and the amount of non-complexed propofol. Thus, it is possible to maintain the amount of cyclodextrin and/or of non-complexed but solvated cyclodextrin derivative present in the solution. By reducing the amount of cyclodextrin and/or cyclodextrin derivative, the risk of nephrotoxicity of the composition of the invention is thus reduced; nephrotoxicity is due to the cyclodextrin or the cyclodextrin derivative itself and the impurities the latter contains. Amongst these impurities, mention may be made of beta cyclodextrin which is used in the synthesis of the derivative.

The use of the salts as mentioned before allows not modifying the pH of the solution of propofol and of cyclodextrin or of cyclodextrin derivative which is used for the preparation of the composition of the invention.

In addition, the use of the salts as mentioned before allows modifying the osmolality of the solution of propofol and of cyclodextrin or of cyclodextrin derivative which is used for the preparation of the composition of the invention, thereby enabling an intravenous injection.

According to one embodiment, the composition of the invention comprises only one type of pharmaceutically-acceptable salt.

Moreover, it turns out that the composition of the invention can be stored at a temperature of 2° C. to 8° C. for at least 6 weeks without observing any decomplexation of the propofol.

The composition of the invention may be in the form of a powder, obtained by freeze-drying, for example, in the form of a gel, preferably having a viscosity enabling it to be injected, or in the form of a solution or a suspension.

The publication entitled “Solubility of cyclodextrins and drug/cyclodextrin complexes” by Saokham, published in 2018 in the journal Molecules, provides an overview of the excipients used to increase the solubility of a given pharmaceutically-active compound by complexation with a cyclodextrin. Thus, various excipients commonly used in pharmaceutical formulations, like organic acids or bases, organic salts (counterions), co-solvents, metal ions and water-soluble polymers, can increase the complexation efficiency of cyclodextrins by stabilisation and solubilisation of the active molecules or molecules of interest/cyclodextrins. This publication also indicates that in the solution, there is still equilibrium between the active molecule in solution, the cyclodextrin alone dissolved in the solution and the cyclodextrin/active molecule complex dissolved in the solution. It also indicates that cyclodextrin complexes tend to aggregate, forming particles likely to precipitate or interfere with filtration thereof, in particular the sterilising filtration of the solution.

Yet, surprisingly, the composition of the invention, when it includes a solvent, can be sterilised by filtration without blocking the pores of the filter membrane. This is the case in particular when the solvent is in the presence of a mineral salt such as, for example, sodium chloride, magnesium chloride, magnesium sulphate, sodium sulphate. The Inventors have not noticed the formation of insoluble complexes when using such salts.

According to a particular embodiment, the composition of the invention consists of propofol, at least one cyclodextrin and/or a cyclodextrin derivative and a pharmaceutically-acceptable salt with the exception of basic and/or acid pharmaceutically-acceptable salts.

Advantageously, irrespective of the embodiment, the composition primarily contains a cyclodextrin derivative, the cyclodextrin being present as an impurity (less than 0.5% by weight). The cyclodextrin derivatives are less nephrotoxic than the cyclodextrins themselves.

The salt or salts may be selected from among:
sodium salts, with the exception of acidic or basic sodium salts, in particular amongst sodium chloride and sodium sulphate;
magnesium salts, with the exception of acidic or basic magnesium salts, in particular, magnesium chloride and magnesium sulphate;
calcium salts, with the exception of acidic or basic calcium salts, in particular, calcium chloride and calcium sulphate.

Preferably, irrespective of the embodiment, the salt is a mineral salt.

Sodium chloride is to be preferred because in water it allows obtaining a physiological serum.

According to a particular embodiment which can be combined with each of the other embodiments, the composition of the invention further contains a pharmaceutically-acceptable solvent, more particularly a polar solvent selected from among alcohols, water, carboxylic acids, amides and mixtures of at least two of these solvents.

Advantageously, irrespective of the embodiment, when it includes a solvent, the composition of the invention has a viscosity suitable for administration by the parenteral route, in particular by the intravenous route, and/or a viscosity enabling sterilisation thereof by filtration.

Advantageously, the composition of the invention consists of the aforementioned solvent, advantageously water, a cyclodextrin derivative and a salt as mentioned before.

Advantageously, irrespective of the embodiment, the cyclodextrin derivative is a water-soluble derivative of cyclodextrin, more preferably of β-cyclodextrin selected from among 2-hydroxyalkyl-β-cyclodextrin and more preferably 2-hydroxypropyl-β-cyclodextrin and from sulphurised alkyl ether derivatives of cyclodextrin and in particular sulphurised alkyl ether derivatives of β-cyclodextrin.

According to a particular embodiment of the injectable form of the composition of the invention, it is in the form of a solution and preferably in the form of a clear solution. In particular and preferably, it may consist of an aqueous solution and advantageously a solution containing only water as a solvent.

Advantageously, the composition of the invention is an aqueous solution of propofol, 2-hydroxypropyl-β-cyclodextrin and sodium chloride and/or magnesium sulphate and/or magnesium chloride and/or sodium sulphate.

Irrespective of the embodiment, the cyclodextrin may advantageously be selected from among β-cyclodextrins and said cyclodextrin derivative is advantageously selected from among the derivatives of β-cyclodextrin, in particular from the group formed by 2-hydroxyalkyl-beta-cyclodextrin, more particularly 2-hydroxypropyl-beta-cyclodextrin and sulphurised alkyl ethers of cyclodextrin of the following formula (II):

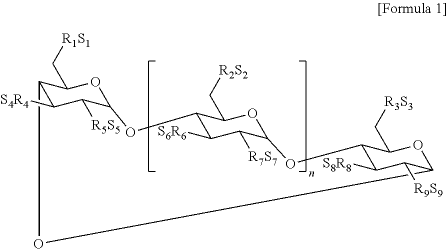

(II) wherein: n is an integer equal to 4, 5 or 6 and the radicals R₁ to R₉ are selected independently of each other amongst an oxygen atom and a group —O—(C₂-C₆ alkylene)—SO₃⁻ and provided that at least R₁ or R₂ is a group —O—(C₂-C₆ alkylene)—SO₃, preferably a group of formula —O—(CH₂)ₘSO₃ wherein m is an integer greater than or equal to 2, less than or equal to 6, preferably greater than or equal to 2 and less than or equal to 4 and the groups S₁ to S₉ are selected independently of each other from among pharmaceutically-acceptable salts.

Advantageously, the injectable composition has a viscosity enabling it to be sterilised by filtration and contains a controlled level of particles formed by aggregation of propofol/cyclodextrin and/or propofol/cyclodextrin derivative complexes whose size exceeds 0.20 μm. Hence, it can be sterilised by filtration. Indeed, according to the aforementioned publication by Saokham, the complexes tend to form particles which might make the solution non-sterilisable by filtration. Hence, it is the merit of the composition of the invention that it can be easily sterilised by filtration when it is in its injectable and in particular liquid form. Thus, it can be easily sterilised in a hospital environment, without any complicated installation.

Advantageously, irrespective of the embodiment, the cyclodextrin derivative is 2-hydroxypropyl-β-cyclodextrin and it advantageously has an average degree of substitution of a glucopyranose unit equal to or greater than 0.50 and equal to or less than 0.71 and in particular equal to 0.69. Such a derivative has proven to be a good complexing agent for propofol, especially in water.

Advantageously, according to an embodiment that can be combined with any one of the aforementioned embodiments, the 2-hydroxypropyl-β-cyclodextrin has a molar mass equal to or greater than 1,179 g and equal to or less than 1,676 g and in particular equal at 1,415.62 g. Combined with the aforementioned average degree of substitution, a 2-hydroxypropyl-β-cyclodextrin is obtained which solubilises propofol well.

Advantageously, in order to reduce the nephrotoxicity of the composition of the invention, it contains only a cyclodextrin derivative that contains less than 0.5% by weight of beta-cyclodextrin. In particular, the cyclodextrin derivative contains less than 0.5% by weight of beta-cyclodextrin. Advantageously, this derivative is 2-hydroxypropyl-β-cyclodextrin.

When the composition of the invention contains a solvent, it advantageously has a pH lower than 8 and higher than or equal to 6 and in particular equal to or higher than 6 and lower than and lower than or equal to 7.45 and/or an osmolatity equal to or greater than 280 mOsmol/kg and less than or equal to 300 mOsmol/kg.

According to an embodiment that can be combined with the other aforementioned embodiments, when it is in solution, in particular in water, the composition of the invention has a pH equal to or higher than 6 and equal to or lower than 8 and in particular comprised between 6.2 and 6.5 (bounds excluded) or comprised between 6.35 and 6.65 (bounds excluded) and an osmolatity equal to or greater than 280 mOsmol/kg and less than or equal to 300 mOsmol/kg. These values may be obtained for sodium chloride and/or magnesium sulphate.

According to a particular embodiment, when it is in solution, in particular in water, the composition of the invention has a pH equal to or higher than 7.35 and equal to or lower than 7.45 and an osmolatity equal or greater than 280 mOsmol/kg and less than or equal to 300 mOsmol/kg. The indicated pH values correspond to the pH values of blood.

Advantageously, the composition of the invention contains an amount of propofol equal to or greater than 0.2 g and in particular an amount of propofol equal to 1.0 g, an amount of cyclodextrin and/or cyclodextrin derivative equal to or greater than 3 g and in particular equal 18 g, 17, 16, 15 g or 14 g, an amount of salt equal to or greater than 0.3 g or 1.5 g.

The solvents, the salts, the propofol, the cyclodextrin(s) and/or the cyclodextrin derivative(s) all have a degree of purity suitable for pharmaceutical use, in particular for parenteral administration.

Advantageously, the composition of the invention has a propofol:β-cyclodextrin and/or β-cyclodextrin derivative molar ratio of 1:<2 and in particular 1:<1.7.

Advantageously, this molar ratio applies to a composition containing a solvent, in particular water, propofol and only a cyclodextrin derivative capable of complexing with propofol, in particular 2-hydroxypropyl-β-cyclodextrin and more particularly 2-hydroxypropyl-β-cyclodextrin defined in the following examples.

Without the Applicants being bound to the following explanation, it seems that the pain of the propofol injection is due to the presence of free propofol in the emulsion. In the case of the present invention, the small amount of free propofol (the propofol is completely complexed in the case of the invention) as well as a pH close to the physiological pH and an osmolality of the solution close to the physiological osmolality allows reducing pain upon injection.

The present invention also relates to a method for manufacturing a composition according to the invention.

Typically, said pharmaceutically-acceptable salt is added under stirring into a mixture of propofol and cyclodextrin and/or cyclodextrin derivative at room temperature or at a temperature equal to or higher than 2° C. and equal to or lower than 20° C., the mixture is mixed so as to obtain a solution, which is preferably clear, then the obtained solution is possibly sterilised by filtration.

The temperature equal to or higher than 2° C. and equal to or lower than 20° C. allows reducing the amount of cyclodextrin/cyclodextrin derivative used to complex a given amount of propofol compared to the amount of cyclodextrin/cyclodextrin derivative used to complex the same given amount of propofol at room temperature. Preferably, the temperature is higher than 2° C. and lower than 15° C., or higher than 2° C. and lower than 10° C., or higher than 2° C. and lower than 8° C.

The propofol complexation is visible to the naked eye. Once all of the propofol is complexed, a clear liquid solution is obtained. If there is still uncomplexed propofol, an opalescent, yellow or white solution or a biphasic mixture is obtained. Adding salt before or during the addition of propofol to the cyclodextrin derivative or cyclodextrin solution enhances the complexation forces. The Inventors have demonstrated that the addition of salt after the cyclodextrin derivative/cyclodextrin increases the stability of the complexation of propofol.

In particular, said cyclodextrin/cyclodextrin derivative is dissolved in said solvent, in particular in water, afterwards propofol is added, then said salt is possibly dissolved in an amount of solvent, in particular water; afterwards an amount of solvent (in particular water) is added allowing obtaining a clear solution (this solvent may also be water). Advantageously, the cyclodextrin and/or the cyclodextrin derivative is dissolved in the solvent, in particular water, before adding the propofol. Hence, a clear cyclodextrin/cyclodextrin derivative solution is obtained before the addition of propofol.

The obtained solution is clear and may be sterilised afterwards by filtration.

Afterwards, the solution of the invention can be stored at room temperature or refrigerated without observing any decomplexation of the propofol.

One could notice that the method of the invention can be easily implemented in a hospital environment without requiring any complex and expensive industrial installation.

### Definitions

In the context of the present invention, an acid salt is defined as being an ionic compound which, in solution, releases H⁺ ions or a counter-ion that is capable of modifying the pH of the solution.

In the context of the present invention, a basic salt is defined as being an ionic compound which, in solution, releases OH⁻ ions or a counter-ion that is capable of modifying the pH of the solution.

In the context of the present invention, a mineral salt is a salt composed of two mineral ions, i.e. including no carbon atom.

The term “cyclodextrin” encompasses alpha, beta and gamma cyclodextrins.

The term “cyclodextrin derivative” encompasses derivatives of alpha, beta and gamma cyclodextrins. A cyclodextrin derivative is a cyclodextrin molecule at least one atom of which has been substituted.

The term “propofol” refers to 2,6-bis(propan-2-yl)phenol.

The term “injectable” refers to a solution/composition that has a suitable viscosity to pass through the needle of an injection syringe commonly used in medicine. A solution, especially a clear solution, may be administered by injection.

In the context of the present invention, the terms “average degree of substitution” refer to the average value of the number of protons substituted by a 2-hydroxypropyl group in each glucopyranose unit of the considered cyclodextrin derivative.

### EXPERIMENTAL PART

### Example

Quantification of Propofol

In all experiments, the amount of propofol has been determined by HPLC coupled with a visible UV spectrophotometer. The analyses have been carried out using a liquid chromatography chain (Thermo Scientific Ultimate 3000) equipped with a diode-array UV detector (DAD 3000). Propofol has been eluted on a C18 column (150×4 mm, grain size: 5 μm) and a Hypersil Gold pre-column (10×5 mm, grain size: 5 μm) maintained at a constant temperature of 25° C., thanks to a Peltier effect oven, (Ultimate TCC 3000). The elution has been carried out at a flow rate of 1 mL/min using a quaternary pump (Ultimate LPG 3400 SD). The mobile phase was composed of a mixture of a 25 mM ammonia buffer, pH 9.2 and acetonitrile (ratio of 52/48% for propofol).

At each analysis, 10 μl of solution are injected, the Propofol peak is obtained at 13.5 minutes and analysed at a wavelength of 270 nm.

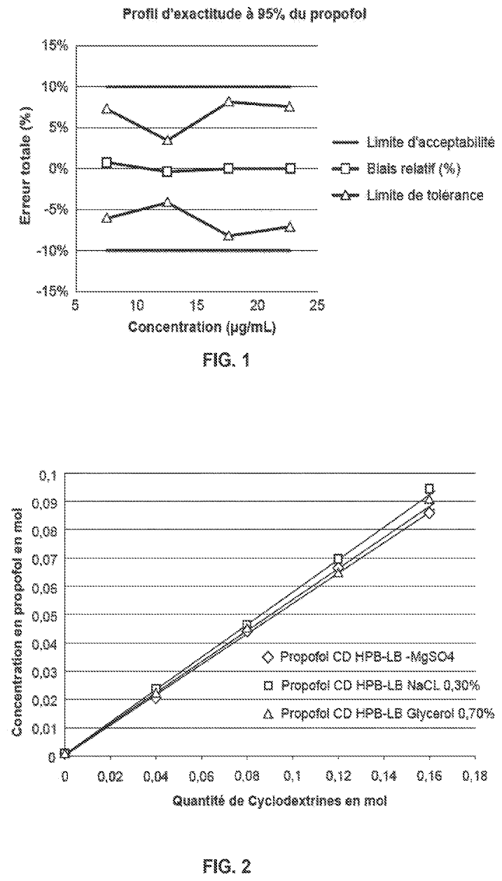

A specific method for determining the concentration and degradation products has been validated. The analytical validation has been carried out in compliance with the French Society of Pharmaceutical Sciences and Techniques [SFSPT]. The validation has consisted in measuring a calibration range at 5 points 5, 10, 15, 20 and 25 μg/ml and 4 quality control points 7.5, 12.6, 17.6 and 22.6 μg/ml repeated 3 times starting from different stock solutions. These analyses have been repeated over 3 consecutive days and 3 different operators for Propofol. A method is validated if the sum of the repeatability risks within the same day and between the three different days is below a predefined error threshold of 10% using a 1 to 5% type Student error test. The obtained results are visible in FIG. 1.

Selection of the Cyclodextrin

Study of the solubility of 2-hydroxypropyl-β-cyclodextrin according to its average degree (or rate) of substitution The used 2-hydroxypropyl-β-cyclodextrin (HPBCD) is commercialised under the trade name of KLEPTOSE® by the company Roquette.

2-Hydroxypropyl-β-cyclodextrins with different average degrees of substitution have been tested. The average degree of substitution corresponds to the average number of propyl groups grafted onto the oxygen atom in the position 2 of each glucose unit that forms β-cyclodextrin. The β-cyclodextrins have 7 glucose units per molecule.

Three 2-hydroxypropyl-β-cyclodextrins having respectively an average degree of substitution equal to or greater than 0.81 and equal to or less than 0.99 (Kleptose® HP), an average degree of substitution equal to or greater than 0.58 and equal to or less than 0.68 (Kleptose® HPB), and an average degree of substitution equal to or greater than 0.50 and equal to or less than 0.71 (Kleptose HPB-LB).

Table 1 below summarises some characteristics of the three 2-hydroxypropyl-β-cyclodextrins.

TABLE 1
Comparative table of the 3 cyclodextrins

| Characteristics | Kleptose HP | Kleptose HPB | Kleptose HPB-LB |
|---|---|---|---|
| Grade | Parenteral/oral | Parenteral/oral | Parenteral |
| Molar mass | 1,501.03 | 1,387.15 | 1,415.62 |
| Colour of the | White | White | White |
| powder | | | |
| Solubility in | Easily | Easily | Easily |
| water | | | |
| European | Yes | Yes | Yes |
| pharmacopoeia | | | |
| conformity | | | |
| Beta-CD impurity | <1.5% by | <1.5% by | <0.5% by |
| | weight | weight | weight |
| Propane diol | <2.5% by | < 2.5% by | <0.5% by |
| impurity | weight | weight | weight |
| Molar substitution | 0.81-0.99 | 0.58-0.68 | 0.50-0.71 |

Throughout the present application, the average substitution rate (MS) is measured by proton NMR. It is calculated from the ratio of the integrations of the H¹ anomeric protons of the macrocycle, and those of the CH₃ group present on the hydroxypropyl group. For the Kleptose HP grade, MS is substantially equal to 0.85 (±2%) and MS=0.65 (±2%) for the HPB grade. For the HPB-LB grade, the average substitution rate is 0.69 (±2%).

The molar mass is calculated according to the value determined for the molar substitution of HPCD, Mw=1135+7×MS×58, 58 being the molar mass of a hydroxypropyl group, 7 corresponding to the number of glucose units and 1,135 being the molar mass of the unsubstituted beta cyclodextrin To make the choice between the 3 types of HPBCD (HP, HPB and HPB-LB), solubility tests have been carried out and interpreted according to the solubility diagrams according to the method of Higuchi and Connors at 25° C. in water. The solubility tests used herein consist in performing measurements of the solubility of propofol at a given constant temperature (herein 25° C.) using different amounts of cyclodextrin at a determined pH value. A large excess of propofol is added to 1.4 ml of the appropriate cyclodextrin solution. The resulting mixtures are vortexed for about 5 min and stirred at a temperature 25±0.5° C. for 24 hours. About 1 ml of the solution is transferred using a Pasteur pipette into an Eppendorf tube. Afterwards, these tubes are centrifuged for 30 min at 13,200 rpm. Afterwards, 40 μl of the aqueous phase are sampled and then diluted to 1/10 in ethanol and to 40/1,000 in a solution of acetonitrile and an ammonia buffer pH 9.2. Afterwards, these samples are filtered through a 0.2 μm filter and then put in vial before analysis by high-performance liquid chromatography with UV detection. The injection volume was 10 microliters (cf. injection loop). The results of the solubility tests have shown a linear solubility, for the three cyclodextrins, of A_{L} type according to the solubility diagram of Higuchi and Connors. The stability constants Kc are estimated from the slope of the line of the phase-solubility diagram according to the following equation: Kc=slope/S0 (1−slope).

The intrinsic solubility (S0) of propofol has been determined directly in solution at a pH between 6 and 7 at a temperature of 25° C.

Table 2 below summarises the intrinsic solubility of propofol in water as well as the stability constants of the complexes formed between the propofol and the various HPβCDs.

TABLE 2
Stability constant of propofol according
to the different cyclodextrins

| | Intrinsic solubility | |
|---|---|---|
| | of propofol in | |
| Type of | water (mol/L)/ | Stability |
| cyclodextrins | (mg/ml) | constant |
| HP | | 1,486 |
| HPB | 0.001073 | 1,145 |
| HPB-LB | 0.19 | 1,288 |

The stability constant Kc is calculated from the slope of the line of the phase-solubility diagram according to the following equation: Kc=slope/S0(1−slope).

One could notice from Table 2 that the stability constants of the complexes formed with the different HPβCDs are relatively close.

Determination of the minimum amount of HPβCD to solubilise 1 g of Propofol.

The solubility tests allow determining the minimum amount of HPβCD necessary to solubilise 1 g of Propofol.

The results are reported in Table 3 below.

TABLE 3
Amount of Cyclodextrins in
grams to solubilise 1 g of propofol

| Type of | Amount |
|---|---|
| cyclodextrins | in gram |
| HP | 13.71 |
| HPB | 13.72 |
| HPB-LB | 13.04 |

In view of the results in Table 3, one could notice that the amount of HPBCD HPB-LB is less to solubilise 1 g of propofol in comparison with the other two cyclodextrins.

However, the solubilisation tests of Kleptose® HPB-LB result in a solubility comparable to Kleptose® HP in mol but with less cyclodextrin in view of Table 3 by weight and less impurity in view of Table 1. Hence, it has been decided to select Kleptose® HPB-LB.

Study of the Excipient

Propofol being a highly lipophilic molecule, the impact of adding different excipients to HPBCD has been studied in order to optimise the solubilised amount of propofol.

Test with a Water-Soluble Polymer Type Excipient

Formulation pre-tests in the presence of PEG 400 have been carried out in the presence of water, propofol and the HPBCD selected in the previous paragraph.

However, the result obtained with PEG 400 still includes 2 distinct, turbid phases, and does not result in a homogeneous and stable formulation. One could deduce that apparently this water-soluble polymer does not seem to improve the solubilisation obtained with the selected HPBCD.

Test with an Excipient in the Form of a Non-Basic and Non-Acidic Salt

Magnesium Sulphate

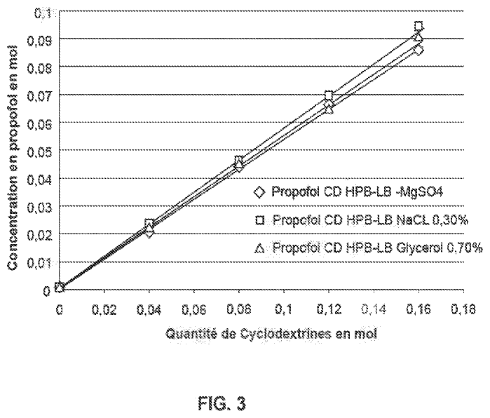

Propofol solubility tests in water in the presence of magnesium sulphate have been carried out according to the previously-described method of Higuchi and Connors. The results are represented in FIG. 3. The used 2-hydropropyl-β-cyclodextrin is still that one selected before (HPB-LB).

One could notice in view of FIG. 3 that the addition of magnesium sulphate does not modify the complexation of propofol and therefore the solubilisation thereof in water for amounts of propofol smaller than or equal to 18 mg/mL.

It should be noted that magnesium is an important ion of the organism, which has many recognised indications and which is very frequently used in anaesthesia, resuscitation and emergency medicine. In the context of this formulation, no toxicity is apparently known at the amounts considered in the formulation.

In addition, magnesium sulphate allows adjusting the osmolality of the formulation and making plasma iso-osmolar.

Sodium Chloride

Propofol solubility tests in water in the presence of sodium chloride have been carried out according to the previously-described method of Higuchi and Connors. The results are represented in FIG. 3. The used 2-hydropropyl-β-cyclodextrin is still that one selected before (HPB-LB).

In view of FIG. 3, one could notice that the addition of sodium chloride does not modify the complexation of propofol and therefore the solubilisation thereof in water for amounts of propofol smaller than or equal to 18 mg/mL.

The results of the solubility tests have been analysed again more particularly with the complexation efficiency (CE), calculated using the equation:

CE=slope/(1−slope)
CE: Complexation efficiency
Slope: Slope of the solubility tests carried out according to the method of Higuchi and Connors

Each solubility test has been carried out 3 times.

The complexation efficiency allows getting rid of the intrinsic variability of propofol, a value that might vary in the case of a lipophilic active ingredient. A high value reflects a better capacity for complexation. The results are disclosed in Table 3a hereinafter:

TABLE 3a
Summary of the
complexation efficiency

| | | Standard |
|---|---|---|
| | Average | deviation |
| HPB | 1.23 | 0.100 |
| HPB-LB Glycerol | 1.24 | 0.112 |
| 0.70% | | |
| HPB-LB (room | 1.26 | 0.035 |
| temperature) | | |
| HPB-LB MgSO₄ 1.5% | 1.32 | 0.071 |
| HPB-LB (2-8° C.) | 1.42 | 0.050 |
| HPB-LB NaCl 0.33% | 1.52 | 0.106 |
| HP | 1.59 | 0.064 |

The analysis of the CEs shows:
A higher CE for cyclodextrin HP, however the latter has more synthetic impurities resulting in that cyclodextrin HPB-LB is preferred
An increased CE in the presence of NaCl and MgSO4
An increased CE when carried out in cold solution

In view of these results, it is clear that the presence of the salts of the invention allows improving the complexation efficiency of propofol.

It has also been established that the presence of these salts allows reducing the necessary amount of cyclodextrin or, and in particular HPBCD necessary to obtain a clear and transparent solution.

Indeed, the Inventors have noticed that to solubilise 1 g of propofol in 100 mL of aqueous solution (10 mg/ml), it is necessary to use about 16 g of HPB-LB at room temperature, and to be able to obtain a clear, transparent and colourless solution. This amount of cyclodextrin may be reduced to 15 g when the solution is obtained between 2° and 8° C. according to the method of the invention, and up to 14 grams in the presence of the salts of the invention.

Hence, the temperature at which the propofol is solubilised has a significant impact on the amount of cyclodextrins CD to be used. This effect is demonstrated in the comparative tests hereinafter, and wherein solutions of 1 gram of propofol in 100 mL (10 mg/ml) of aqueous solution have been prepared:
i) With water between 2 and 8° C. (upon mixing),
   In a solution of 14 g of HPB-LB, a clear, colourless solution is obtained but turns yellow rapidly during storage, indicating a decomplexation of propofol.
   In a solution of 15 g of HPB-LB, a clear and colourless solution is obtained.
ii) With water at room temperature (upon mixing)
   In a solution of 14 g of HPB-LB, a clear and slightly yellow solution is obtained, indicating an incomplete complexation of propofol.
   In a solution of 15 g of HPB-LB, a slightly turbid, colourless solution is obtained but with visible particles.
   In a Solution of 16 g of HPB-LB, a clear and colourless solution is obtained.

In a preferred embodiment, the invention suggests preparing the propofol composition at a temperature between 2 and 8° C., and using sodium chloride salts, and sodium sulphate, magnesium chloride, magnesium sulphate, calcium chloride, and calcium sulphate to reduce the amount of cyclodextrin or its derivative necessary to solubilise propofol, as well as improving complexation thereof.

The advantages of the invention are observed in the results of the comparative solutions hereinafter, and obtained to solubilise 1 gram of propofol in 100 mL of aqueous solution (10 mg/mL) at a temperature between 2 and 8° C.:
Solution of 15 g of HPB-LB without excipient, a clear and transparent solution is obtained. The osmolarity is 126 mOsm/kg.
Solution of 14 g of HPB-LB without excipient, a clear and transparent solution is obtained but with a slight yellow colouration, indicating an incomplete complexation. This solution becomes turbid and yellowish in less than one week of storage. The osmolarity is 114 mOsm/kg.
Solution of 14 g of HPB-LB and 0.4% NaCl, a clear, transparent, colourless and stable solution is obtained during storage. The osmolarity is 264 mOsm/kg.

The positive effect on improving the complexation of propofol is obtained starting from a concentration of at least 0.3% by weight of added salt, and preferably at least 0.4% by weight of salt, in particular NaCl. The mass percentage of salt to be used may also be optimised according to the selected salt. A higher concentration of salt may be used, in particular in order to reach an osmolality of 280 mOsm/kg being optimum for the clinical use of this drug by the intravenous route. Indeed, the osmolality of an injectable drug is optimum when the isotonia is equal to that of plasma (280-300 mOsmol/L), which enables use by the peripheral route (currently used in the clinic). In case of injection of a hypo-osmolar solution, the red blood cells swell and burst, this is haemolysis. In case of injection of a hyperosmolar solution, the red blood cells become deformed: the external environment is hypertonic, resulting in the release of water from the red blood cells and therefore a plasmolysis phenomenon.

The invention also suggests using a polar co-solvent, such as alcohols in order to further reduce the amount of cyclodextrin necessary to solubilise and stabilise the propofol. Such a composition may be obtained as follows:

At room temperature, a solution of 14 g of HPB-LB has been made with a mixture of 50% ethanol at 96 and 50% water as solvent, to solubilise 1 g of propofol, in 100 mL (10 mg/ml).

The solubilisation of the CDs is performed in 50 mL of ethanol; the solubilisation is longer than in water, but enables a solubilisation of all HPB-LB in a few minutes. Afterwards, one gram of propofol then 50 mL of water are added; the whole is steered in vortex for 15 min.

The solubilisation of propofol in the solution of 50% ethanol and 50% water results in a clear, colourless and transparent solution.

The same result (clear, colourless and transparent solution) is found with 13 g of HPB-LB and 50% ethanol. With the use of an ethanol solvent at 50%, the amount of CD is reduced to 13 g for 1 g of propofol for 100 mL. This solution cannot be used for an intravenous injection. It is all the same possible to reduce the amount of cyclodextrins by the use of a polar co-solvent and in combination with the addition of salts according to the invention.

The results of the different comparative tests of propofol 10 mg/ml solutions described hereinbefore are disclosed in Table 3b below:

| | Amount of CD for 100 mL | | | | | |
|---|---|---|---|---|---|---|
| NaCl | 14 g | | | 15 g | | |
| concentration | Aspect | Osmolarity | pH | Aspect | Osmolarity | pH |
| 0% | L => Turbid | 114 | 5.9 | Clear | 126 | 6.31 |
| 0.40% | Clear | 264 | 4.84 | Clear | 283 | 5.15 |
| 0.80% | Clear | 415 | 5.06 | | | |
| 50% OH | Clear | * | 5.77 | | | |
| 2% OH + 0.4% | Clear | 727 | 5.06 | | | |
| NaCl | | | | | | |

Table 3b Summary of cold solubilities at 8° C. according to different amounts of cyclodextrins 14 and 15 g, without excipient, and with excipient NaCl 0.4%, 0.8%, in the presence of 50% ethanol; 2% ethanol and 0.4% NaCl. (*: Osmolality not measurable). The unadjusted pH and osmolarity obtained with the different solutions are also indicated.

Test with Addition of Glycerol

Propofol solubility tests in water in the presence of glycerol and 2-hydropropyl-β-cyclodextrin have been carried out according to the previously-described method of Higuchi and Connors. The results are represented in FIG. 3. The used 2-hydropropyl-β-cyclodextrin is still that one selected before.

One could notice in view of FIG. 3 and Kc in the presence of glycerol that the latter reduces the complexation of cyclodextrins in the presence of propofol.

Hence, the addition of glycerol is not considered.

Table 4 below summarises the intrinsic solubility of propofol in water in the presence of excipients and the stability constants of cyclodextrin HPB-LB in the presence of different excipients determined according to the method of Higuchi and Connors.

TABLE 4
Intrinsic solubility of propofol in combination
with excipients and stability constant of
propofol according to the excipient

| | Intrinsic solubility | |
|---|---|---|
| | of propofol + | |
| | HPB-LB in | |
| | Water + excipient | Stability |
| excipient | (mol/L)/(mg/ml) | constant |
| NaCI | 0.001096/(19.52) | 1,269 |
| MgSO₄ | 0.00096/(17.01) | 1,238 |
| Glycerol | 0.00133/(23.67) | 934 |

### Formulation Examples

Formulation examples are given in tables 5 and 6 hereinafter:

All of the following formulations allow obtaining clear solutions.

The manufacturing protocol for the solutions is as follows:
Weighing the HPBCD
Dissolution in 75% of the final volume of water for injectable preparations
Mix until a clear and transparent solution is obtained
Addition of propofol drop-by-drop
Addition of non-basic and non-acidic salt
Addition of water for injectable preparations Q.S. 100 mL
Mix under stirring for at least 10 min until a clear solution is obtained
Storage of the preparation for several hours.
Sterilising filtration on a membrane having pore size smaller than 0.22 μm
Packaging in sterile vials.

A clear, colourless solution with a hydrocarbon odour is obtained.

Particles Visible to the Naked Eye: Absent

The Propofol 1% injectable solution is stored at room temperature or refrigerated. Preliminary tests suggest that this method of preservation is appropriate.

The steps of dissolving the HPBCD and the salt may be implemented at a temperature equal to or higher than 2° C. and equal to or lower than 20° C. in order to reduce the amount of cyclodextrin, and preferably lower than 15° C., or lower than 10° C.

In all examples, the HPBCD is that one selected before.

Formulation 1: in the presence of sodium chloride-protocol implemented at a temperature equal to or higher than 2° C. and equal to or lower than 20° C.

TABLE 5

| Compound | Amount (g) |
|---|---|
| Propofol | 1 |
| HPβCD | 14 |
| Sodium | 0.4 g |
| chloride | |
| Distilled | Q.S. to reach |
| water | 100 mL |

The pH of the solution is comprised between 6.35 and 6.65, osmolality between 280 and 300 mOsmol/kg.

Formulation 2: In the presence of magnesium sulphate protocol implemented at a temperature equal to or higher than 2° C. and equal to or lower than 20° C.

TABLE 6

| Compound | Amount (g) |
|---|---|
| Propofol | 1 |
| HPβCD | 14 |
| Magnesium | 1.5 g |
| sulphate | |
| Distilled | Q.S. to reach |
| water | 100 mL |

The pH of the solution is comprised between 6.2 and 6.5, osmolality between 280 and 290 mOsmol/kg (bounds included).

All of the solutions of the invention have been stored for 6 weeks in a refrigerator, at a temperature equal to or higher than 2° C. and equal to or lower than 8° C. Within 6 weeks, the solutions are still clear, which indicates that the propofol is still solubilised by complexation with 2-hydropropyl-β-cyclodextrin. Hence, the solutions are suitable for storage without decomplexation of the propofol.

This effect of reinforcing the complexation of propofol with cyclodextrin also seems to be related to the method of preparation of the composition. In particular, the effect of adding salts before or after the complexation of propofol with cyclodextrin has been demonstrated. The results hereinafter have been obtained after 1 week of storage of a composition according to the invention, initially clear and colourless, and into which the salts are added before or after mixing the propofol with the cyclodextrin:
Solution of 14 g of HPB-LB and 1% of propofol with addition of NaCl before mixing propofol HPB-LB, a clear and transparent solution is obtained but with a slight yellow colouration.
Solution of 14 g of HPB-LB and 1% of propofol with addition of NaCl after mixing, a clear, transparent, colourless solution is obtained.

The results show that the addition of salts after solubilisation of propofol with cyclodextrin or cyclodextrin derivative allows guaranteeing the stability of the complexation of propofol-cyclodextrin over time. Hence, the composition of the invention is suitable for storage thereof without the need for additional excipients.

1. A pharmaceutical composition comprising:
propofol,
at least one cyclodextrin and/or one cyclodextrin derivative,
at least one pharmaceutically-acceptable salt selected from the group consisting of sodium chloride, sodium sulphate, magnesium chloride, magnesium sulphate, calcium chloride, and calcium sulphate,
wherein the pharmaceutical composition is in the form of a clear solution having a viscosity enabling sterilisation thereof by filtration; and
said solution is colourless and suitable for storage without de-complexification of propofol.

2. The composition according to claim 1, wherein the propofol and said cyclodextrin or cyclodextrin derivative are present in a molar ratio of 1:≤2.

3. The composition according to claim 1, wherein the propofol and said cyclodextrin or said cyclodextrin derivative are present in a mass ratio 1:<15.

4. The composition according to claim 1, wherein said clear solution is obtained by adding said salt into a solution including said propofol and said cyclodextrin at a temperature higher than 2° C. and lower than 20° C., and preferably lower than 15° C.

5. The composition according to claim 1, further comprising a pharmaceutically-acceptable solvent, wherein the pharmaceutically-acceptable solvent is a polar solvent selected from a group consisting of alcohols, water, carboxylic acids, amides and mixtures of least two of these solvents.

6. The composition according to claim 1, wherein the viscosity is suitable for parenteral administration.

7. The composition according to claim 1, wherein said cyclodextrin is selected from a group of β-cyclodextrins and/or in that said cyclodextrin derivative is selected from a group of derivatives of β-cyclodextrin, in particular from the group formed by 2-hydroxyalkyl-beta-cyclodextrin, more particularly 2-hydroxypropyl-beta-cyclodextrin and sulphurised alkyl ethers of cyclodextrin of the following formula (II): 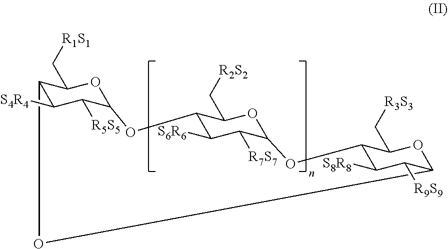
wherein is an integer equal to 4, 5 or 6 and the radicals R₁ to R₉ are selected independently of each other amongst an oxygen atom and a group —O—(C₂-C₆ alkylene)—SO₃⁻ and provided that at least R₁ or R₂ is a group —O—(C₂-C₆ alkylene)—SO₃—, preferably a group of formula —O—(CH₂)ₘSO₃ wherein m is an integer greater than or equal to 2, less than or equal to 6, preferably greater than or equal to 2 and less than or equal to 4 and the groups S₁ to S₉ are selected independently of each other from among pharmaceutically-acceptable salts.

8. The pharmaceutical composition according to claim 1, wherein it contains 2-hydroxypropyl-β-cyclodextrin whose average degree of substitution of a glucopyranose unit is equal to or greater than 0.50 and equal or less than 0.71 and in particular equal to 0.69.

9. The pharmaceutical composition according to claim 1, wherein it contains 2-hydroxypropyl-β-cyclodextrin with a molar mass equal to or greater than 1,179 g and equal to or less than 1,676 g and in particular equal to 1,415.62 g.

10. The pharmaceutical composition according to claim 1, wherein it contains only a cyclodextrin derivative and in that said cyclodextrin derivative contains less than 0.5% by weight of beta-cyclodextrin.

11. The pharmaceutical composition according to claim 1, wherein it has a lower than an higher than or equal to 6 and in particular equal to or higher than 6 and lower than or equal to 7.45 and/or an osmolatity equal to or greater than 280 mOsmol/kg and less than or equal to 300 mOsmol/kg.

12. A method for manufacturing a composition according claim 1, wherein said salt is added under stirring into a mixture of propofol and cyclodextrin and/or cyclodextrin derivative at room temperature or at a temperature equal to or higher than 2° C. and equal to or lower than 20° C., then in that the obtained solution is possibly sterilised by filtration.

13. The method according to claim 12, wherein the temperature is lower than 10° C., and preferably lower than 15° C.
